(19) United States
(12) Patent Application Publication
(10) Pub. No.: US 2005/0154292 A1
(43) Pub. Date: Jul. 14, 2005

(54) Method and control device to operate a magnetic resonance tomography apparatus

(76) Inventor: Martin Tank, Heidelberg (DE)

Correspondence Address: SCHIFF HARDIN, LLP;PATENT DEPARTMENT, 6600 SEARS TOWER, CHICAGO, IL, 60606-6473, US

(21) Appl. No.: 11/006,993

(22) Filed: Dec. 8, 2004

(30) Foreign Application Priority Data
Dec. 8, 2003 (DE) 103 57 203.1

(51) Int. Cl. A61B 5/05

(52) U.S. Cl. 600/410

(57) ABSTRACT

In a method and control device for the operation of a magnetic resonance tomography apparatus, initially an anatomical normal model whose geometry is variable is selected for an examination subject to be examined dependent on a diagnostic inquiry. Then a number of overview images of a region of the examination subject are obtained, with various overview scan parameters with which the acquisition of the overview images is controlled being established dependent on the selected anatomical normal model. In the slice image data of the acquired overview images, a target structure is determined and the normal model is individualized for adaptation to the determined target structure. Scan parameters for control of the magnetic resonance tomography apparatus for acquisition of subsequent slice images dependent on the selected normal model and a diagnostic inquiry are then selected and individualized corresponding to the individualized normal model. The acquisition of the slice image exposures the ensues on the basis of these individualized scan parameters.

### BACKGROUND OF THE INVENTION

1. Field of the Invention

The invention concerns a method to operate a magnetic resonance tomography apparatus (MRT apparatus). The invention also concerns a corresponding control device to operate a magnetic resonance tomography apparatus.

2. Description of the Prior Art

The results of magnetic resonance tomography examinations are typically multiple series with a number of section images (slice images) of the examination subject of interest, for example the head, a knee, the pelvis or a specific organ of a patient, or of a more expanded body region of the patient. The planning of the examination, i.e. the establishment of various scan parameters (such as, for example, the position and number of the slice stacks or of the individual slices, the separations of the slices from one another, the volumes, the observation window and the size of the measurement matrix, as well as the saturation regions, etc.) typically ensues interactively through an operator of the apparatus. Generally, a measurement initially begins with the acquisition of overview images (also called “localizer scans” or “scout scans”) of the entire patient or at least of a wide range of the region of interest. Based on these overview images, the slices/volumes to be examined are then defined by the operator with the aid of a graphical user interface and the other scan parameters are established. For this purpose, the control device of the magnetic resonance tomography apparatus typically executes control software. This planning normally makes a correlation to prominent anatomical structures detected in the overview images and is thus dependent on the respective operator. This leads to reproducible examinations being practically impossible, so that an exact monitoring of diseases is made more difficult since the slice orientations and slice positions corresponding to one another in similar examinations implemented at different points in time can deviate significantly from one another. A further problem is that during the entire examination time a person must be provided for the operation of the device. This person typically can attend no other tasks during the examination time. High requirements are placed on the qualifications of the of the operator, since the diagnostic significance of the acquired exposures is significantly dependent on the positioning of the slices to be measured and the (if applicable) necessary saturation slices, as well as on other scan parameters to be set. Pre-prepared measurement protocols have been provided on many control devices that contain various parameters for specific diagnostic inquiries, examinations, with defaults to various predetermined parameters. Nevertheless, these prepared measurement protocols must be respectively adapted to the individual case, and the entry of a number of further scan parameters is necessary in the framework of the interactive planning.

An objective and completely automatic method to determine the significant scan parameters in order obtain reproducible examination results and in order to optimize the workflow is therefore desirable.

Various proposals have been made in order to automate the planning of magnetic resonance examinations.

Thus, for example, German OS 101 60 075 and United States Patent Application Publication No. 2002/1098447 describe various possibilities for obtaining, series examinations dependent on preceding examinations optimized for time and implemented automatically as much as possible.

Furthermore, U.S. Pat. No. 6,529,762 (corresponding to German OS 199 43 404) describes a method in which anatomical landmarks are identified in the overview images and the measurement parameters for subsequently magnetic resonance measurements are then established using these landmarks. This ensues by a comparison of the acquired overview images with stored reference overview images. The current overview images are adapted to the reference overview images for this purpose. This method assumes, however, that sufficient reference images are available which are suited for comparison with the current overview images.

In U.S. Pat. No. 6,195,409, an alternative method is described in which the overview images are first analyzed in order to detect important structural information (such as, for example, size, location and orientation) about the examination subject of interest and possible partial subjects, which then leads to an abstract, schematic specification (known as a “model”) of the subject of interest. This abstract model contains information about vertices of the examination subject and information about the stability of the connections between these vertices as geometric information. This abstract model of the examination subject is then adapted to a pattern model. Different pattern models are available for various adaptation levels. A head pattern model is composed of the pattern models “rectangular box”, “skin surface model”, “brain model” and “model of an inner brain structure”. A problem in all of these methods is the adaptation of the model to the geometric information acquired from the overview exposures. It is clear that the adaptation quality is strongly dependent on the type and the quantity of the information acquired from the overview exposures. The creation of the localizer scans is an important criterion for the overall quality of the adaptation process and the control of the further examination based thereon.

### SUMMARY OF THE INVENTION

An object of the present invention is to provide an alternative to the above-described methods and control devices that enables, in an optimally safe and simple manner, control of a magnetic resonance tomography apparatus that is substantially fully automatic and can be reproduced at any time during an examination.

This object is achieved by a method according to the invention that, in a manner different from the conventional methods, begins with the selection of an anatomic normal model, whose geometry is variable for an examination subject to be examined dependent on the diagnostic problem. This means, for example, that a skull model is selected given an examination of the head of a patient or a knee model is selected given a knee examination. This model can be composed of a number of model parts, for example of a model bone structure which in turn is separated into the individual parts of the respective examination subject. Thus, for example, a skull bone model can include the parts “frontal bone” (os frontale), “right parietal bone” (os parietale dexter), “left parietal bone” (os parietale sinister), “visceral cranium” (viscerocranium), “occipital bone” (os occipitale), “base of the skull” (basis cranii interna) and “mandible” (mandibula).

A number of overview images of a region of examination subject is subsequently produced. Various overview image scan parameters, using that the measurement of the overview images is controlled, are established dependent on the selected anatomical normal model. A target structure is then determined in the slice image data of the measured overview images—if applicable, dependent on the diagnostic question and/or dependent on the normal model. An automatic individualization of the normal model subsequently ensues for adaptation to the determined target structure. Since the overview scan parameters are established dependent on the respective normal model, it is ensured that a sufficient number and the correct type of the overview images are generated for the respective normal model, such that the target structure that can be determined therein contains sufficient information in order to be able to correctly adapt the normal model to the target structure with the greatest possible safety.

Scan parameters for the control of the magnetic resonance tomography apparatus are then selected dependent on the selected normal model and on the diagnosis question. These scan parameters relate to the selected normal model. Therefore an individualization of the selected scan parameters is initially implemented corresponding to the individualized normal model. Finally, the measurement of the slice image exposures ensues based on these individualized scan parameters.

Since the measurement of the overview images and the determination of the target structure ensues dependent on the selected normal model in the proposed inventive method, it is ensured with significantly higher safety than in conventional methods that the individualization of the normal model (on which ultimately the quality of the determination of the correct scan parameters is dependent) is implemented in a correct manner. Therefore the quality and primarily the reproducibility of automatic measurements are significantly increased via the inventive method.

To implement this method, outside of a typical interface for control of the magnetic resonance tomography apparatus, an inventive control device to operate a magnetic resonance tomography apparatus requires a storage device with a number of anatomical normal models with variable geometry in order to measure a number of slice image exposures corresponding to scan parameters predetermined by the control device. The normal models are respectively associated with various examination subjects. Moreover, a first selection unit (in order to select one of the anatomical normal models for an examination subject to be examined dependent on a diagnostic question) and an overview image determination unit are necessary in order to control the magnetic resonance tomography apparatus to measure a number of overview images of a region of the examination subject using overview scan parameters that are predetermined dependent on the selected anatomical normal model. Furthermore, a target structure determination unit to determine a target structure in the slice image data of the measured overview images as well as an adaptation unit are necessary in order to individualize the selected normal model for adaptation to the determined target structure. Furthermore, a second selection unit is necessary to select scan parameters for control of the magnetic resonance tomography apparatus for a measurement of subsequently slice images dependent on the selected normal model and on the diagnostic question, as well as a parameter individualization unit which likewise individualizes the selected scan parameters corresponding to the individualized normal model.

Moreover, the control preferably should also include all further typical components that are necessary for operation of a magnetic resonance tomography apparatus such as, for example, an interface for image data acquisition and to prepare the image data as well as a console or another user interface via which the user can, for example, also enter the diagnostic inquiry.

Preferably, after the individualization of the normal model it is first checked whether the remaining deviations of the individualized normal model from the target structure lie below a predetermined threshold. Otherwise the method is canceled. As before, the further examinations must be manually planned or controlled. Via this examination it is safely prevented that, in cases in which the model is not adapted well enough to the overview images or to the target structures detectable therein, an automatic planning and examination control is nevertheless implemented and thus faulty further images are prevented from being generated that can possibly be falsely interpreted in a later diagnosis. Instead of a test of the remaining deviation of the individualized normal model from the target structures, it is also possible, for example, to then terminate when no predetermined deviation limit is achieved in the individualization after a specific time. For this, the inventive control device requires a corresponding testing unit.

The various normal models preferably are mutually stored with the overview scan parameters associated with them. It is thus feasible to store the normal models and the associated overview scan parameters in a databank or in databanks networked with one another. “Mutually stored” means that, for example, pointers or similar which refer to storage regions in which the overview scan parameters are then to be found or vice versa are stored with the normal models.

All parameters for determination of the position (i.e. for determination of the position and orientation) of the individual slices, for determination of the separations of the slices from one another and for determination of the number and also of the type of the overview images preferably belong to the overview scan parameters. “Scan parameters for determination of the type of the overview images”, means parameters with which, for example, the type of the pulse sequence used is set, etc. Normally gradient echo protocols are used for acquisition of overview exposures due to the higher measurement speed. For orthopedic questions, however, spin-echo protocols often are used for the overview exposures. For heart examinations fast single-shot protocols are used due to the significant movement artifacts that would occur otherwise.

The individualization of the anatomical normal model, i.e. the adaptation to the target structure, can fundamentally be implemented with an arbitrary suitable individualization method. The idea of the individualization of an anatomical model generally can be formulated in a simplified manner, such that a geometric transformation—corresponding to a three-dimensional transformation in a three-dimensional model—is sought that optimally adapts the model to an individual data set. All information that can be associated with the geometry of the model is thereby likewise individualized. In medical image processing, such a method for determination of optimal transformation parameters is also designated as a registration or matching method. Differentiation is typically made between what are known as rigid, affine, perspective and elastic methods, depending on which geometric transformation is used. For mathematical processing of the individualization problem, a deviation function preferably is used that describes the deviation of the arbitrarily transformed model from the target structure. The type of the deviation function depends on the respective type of the anatomical normal model used. This enables a simple, complete, automatic individualization of the model by minimization of the deviation value, i.e. minimum of the deviation function is controlled in the adaptation.

In order to optimally quickly find a minimal value of the deviation function, a multi-stage method preferably is used. For example, in a three stage method the model can first be roughly adapted with the aid of a fitting positioning, i.e. translation, rotation and a scaling. In a second step, a volume transformation can then be implemented in order to achieve a better calibration. In a third stage, a fine-tuning is subsequently implemented in order to locally, optimally adapt the model to the structure.

The automatic adaptation can ensue entirely in the background, such that the operator can address other tasks and, in particular, can also process other image data or control further measurements in parallel on the appertaining console of the image processing system. It is also possible that during the automatic method, the process is permanently shown, for example on a screen (or a portion of a screen), such that the user can monitor the progress of the adaptation process. Therefore the actual value of the deviation function preferably is displayed to the operator. In particular it is also possible to permanently display the deviation values on the screen, for example in a task bar or the like, while the rest of the user interface is free for other tasks of the operator.

The possibility exists for the operator to intervene in the automatic adaptation process as needed and to manually adjust individual model parameters. The current deviation value is displayed to the operator, such that the operator immediately sees, in the variation of the appertaining model parameters, whether and to which degree the geometry deviations are reduced by the operator's actions. It is also possible to determine individual deviation values for each model parameter and to display this instead of an overall deviation value or in addition to this. A typical example for this is the representation of the target structure and/or the normal model to be adapted or at least of parts of these subjects on a graphical user interface of a terminal. The user can adapt a specific model parameter—for example the distance between two points on the model—with, for example, the aid of the keyboard or with the assistance of a pointing device such as a mouse or the like. By means of a running bar or in a similar optical, easily recognizable manner, it is then indicated to the user to what extent the deviations are reduced the user's actions. In particular, the overall deviation of the model is shown as well as the deviations with regard to the adaptation of the concrete, current model parameter (for example given a separation of two points in the model representing the separation between the appertaining points in the target structure).

The usable, digital, anatomical normal models in principle can be constructed in various manners. One possibility is, for example, modeling anatomical structures on a voxel basis, but special software that is normally expensive and uncommon is necessary for the editing of such volume data. Another possibility is modeling with items known as “finite elements”, whereby normally a model is assembled from tetrahedrons. Special and expensive software, however, also is necessary for such models. A simple modeling of anatomical boundary surfaces by triangulation is relatively widespread. The corresponding data structures are supported by many standard programs from this field of computer graphics. According to this principle, assembled models are designated as models known as surface-oriented models. This concerns the smallest common denominator of the modeling of anatomical structures, since corresponding surface models can be derived both from the first cited volume models by triangulation of the voxels and from a conversion of the tetrahedrons of the finite elements method into triangles. It therefore lends itself to use as normal models assembled on a triangle basis, surface-oriented models. The models are generated in the simplest and most cost-effective manner with this method. Models already generated in another form, in particular the cited volume models, are transferred via a suitable transformation, such that recreation of a corresponding model is not necessary.

In order to newly create such surface models, slice image exposures can be segmented with, for example, a classical, manual method. Ultimately, the models can be generated from the thusly-acquired information about the individual structures, for example individual organs. In order to obtain human bone models, for example, a human skeleton can be measured using laser scanners or can be scanned and segmented as well as triangulated with a computed tomography scan.

In the inventive method, a normal model is preferably used in which the model parameters are hierarchically ordered with regard to their influence on the anatomical overall geometry of the model. The individualization of such a hierarchically parameterized normal model then ensues in a number of iteration steps. With an increasing number of iteration steps, the number of the model parameters (simultaneously adjustable in the respective iteration step, and thus the number of the degrees of freedom in the model variation) is increased corresponding to the hierarchical order of the parameters. By this method it is ensured that, in the individualization, the model parameters which have the greatest influence on the anatomical overall geometry are adjusted first. The subordinate model parameters which only influence a part of the overall geometry are only then adjustable. An effective and consequently timesaving procedure is thus ensured in the model adaptation, independent of whether the adaptation is implemented completely automatically or whether an operator manually intervenes in the adaptation method. Given a (partially) manual method, this can, for example, be realized by the individual model parameters only being provided to the operator for variation (for example by means of a graphical user interface) in each iteration step according to their hierarchical order.

The model parameters preferably, are respectively associated with a hierarchy class. This means that different model parameters can possibly be associated with the same hierarchy class, since they have an approximately identical influence on the anatomical overall geometry of the model. All model parameters of a specific hierarchy class can then be added to the adjustment again in a specific iteration step. In a next iteration step, the model parameters of the subordinate hierarchy class are then added, and so on.

The association of a model parameter with a hierarchy class can ensue on the basis of a deviation in the model geometry which occurs when the appertaining model parameter is changed by a specific value. In a preferred version of the method, specific ranges of deviations, for example numerical deviation intervals, are associated with various hierarchy classes. This means that, for example for classification of a parameter in a hierarchy class, this parameter is changed and the resulting deviation of the geometrically changed model from the initial state is calculated. The degree of deviation depends on the type of the normal model used. It is necessary only that a precisely defined degree of deviation be determined, which as precisely as possible quantifies the geometry modification of the model before and after variation of the appertaining model parameter in order to ensure a realistic comparison of the influence of the various model parameters on the model geometry. For this purpose, a uniform increment preferably is used for each parameter type, for example for displacement parameters in which the separation between two points is varied, or for angle parameters in which an angle is varied between three points of the model, in order to be able to directly compare the geometry influence. The parameters are then simply classified into the hierarchy classes by a specification of numerical intervals for this degree of deviation. Given the use of surface models generated on a triangle basis, the deviation between the unmodified normal model and the modified normal model after variation of a parameter is preferably calculated on the basis of the sum of the geometric separations of corresponding triangles of the models in the various states.

Preferably, at least the model parameters in which variation of the normal model is globally modified are classified in an uppermost hierarchy class whose model parameters are immediately adjustable in a first iteration step. Included for this are, for example, the total of nine parameters of the rotation of the entire model around the three model axes, the translation along the three model axes and the scaling of the entire model along the three model axes.

The hierarchical classification of the individual model parameters can in principle ensue during the individualization of the model. For example, in each iteration step it is initially checked which further model parameters have the largest influence on the geometry, and then these parameters are added. Since a significant computational effort is associated with this, the classification of the model parameters in hierarchical order particularly preferably ensues beforehand, for example even at the generation of the normal model, however at least before the storage of the normal model in a model databank. This removal of the hierarchical arrangement of the model parameters into a separate procedure for the generation of a normal model has the advantage that for each model the calculation of the hierarchical order of the model parameters must only be calculated once, and thus valuable calculation time can be saved during the segmentation. The hierarchical order also can be mutually saved with the normal model in a relatively simple manner, for example by the parameters being stored ordered in hierarchy classes or with corresponding markers or similar linked in a file header or at another normalized position in the file, which also contains the further data of the appertaining normal model.

In a preferred embodiment, the model parameters are respectively linked with a position of at least one anatomical landmark of the model, such that the model exhibits an anatomically reasonable geometry for every parameter set. Typical examples for this are the global parameters such as rotation or translation of the overall model, in which all model parameters are correspondingly, fittingly modified to one another in terms of position. Other model parameters are, for example, the distance between two anatomical landmarks or an angle between three anatomical landmarks, for example or determination of a knee position.

Such a coupling of the model parameters to medically, reasonably selected anatomical landmarks has the advantage that a diagnostic conclusion is always possible after the individualization. In the anatomical subject literature, the positions of such anatomical landmarks are additionally described exactly. With such a procedure, the implementation of the individualization is made easier since a medically trained user, for example a doctor or an MTA, is familiar with the anatomical landmarks and these significantly determine the anatomy.

There are various possibilities for the automatic determination of the target structure of the partial subject to be separated in the slice image data. One alternative is to apply what is known as the “threshold method”. This method functions in the manner that the intensity values of the individual voxels, i.e. the individual 3D image points, are compared with a fixed threshold. If the value of the voxel is above the threshold, this voxel is assigned to a specific structure. With magnetic resonance exposures this method is primarily applicable in contrast agent examinations or for identification of the skin surface of a patient. This method is generally not suitable for detection of other tissue structures. In a preferred version, the target structure is therefore at least partially determined by means of a contour analysis method. Such contour analysis methods operate on the basis of the gradients between adjacent image points. The most varied contour analysis methods are known to the average man skilled in the art. The advantage of such contour analysis methods is that the methods can be used in a stable manner.

In a further embodiment of the inventive method, it is also possible to automatically classify the examination subject. Thus it can be automatically established whether further examinations are necessary and, if so, which examinations are implemented. This embodiment also allows submitting the classification to the operator only as a suggestion, such that he can then accept the suggestion or reject it.

Such an automatic classification of an examination subject can ensue in the manner that specific anatomical structures as well as the deviations of these structures from an individualized comparison model or, respectively, comparison model part are automatically determined in the measured slice image data. Given the individualization of this comparison normal model, it must be ensured that only transformations are implemented such that the geometry of the comparison normal model, or of the appertaining normal model part itself exhibits no pathologies. Pathologies of the examined anatomical structures thus can be automatically established in a simple manner, and then further examinations can be automatically established based on this. The determined deviations also can be graphically visualized together with the anatomical structures, for example be marked on a screen for the operator. Additionally, such deviations can be indicated to the operator via an acoustic signal.

The first selection unit, the overview images determination unit, the target structure determination unit, the adaptation unit, the second selection unit for the selection of control parameters and the parameter individualization unit of the inventive control device preferably are realized in the form of software on a processor of a programmable control device. This control device should moreover comprise as hardware components, among other things, the interface for the activation of the magnetic resonance tomography apparatus as well as a storage device in order to store the anatomical normal models, preferably together with the overview scan parameters and the further scan parameters for the examinations. This storage device does not necessarily have to be an integrated part of the control device. It is sufficient for the image computer to be able to access an appropriate external storage device or a number of distributed storage devices.

A realization of the inventive method in the form of software has the advantage that existing storage devices can also be relatively simply, correspondingly upgraded via suitable updates.

### DESCRIPTION OF THE DRAWINGS

FIG. 1 is a schematic block diagram of an exemplary embodiment of a magnetic resonance tomography apparatus with an inventive control device.

FIG. 2 is a flowchart of an embodiment of the inventive method.

FIG. 3 is a flowchart of a preferred method for model individualization as used in accordance with the invention.

FIG. 4A is a representation of a surface model of a human skull with five sagittal slice planes as used in accordance with the invention.

FIG. 4B is a representation of the surface model according to FIG. 4A, but with five transversal slice planes.

FIG. 5 is a representation of the target structure of a human skull on the basis of slice image data as used in accordance with the invention.

FIG. 6a is a representation of the target structure according to FIG. 5 with an unadapted surface normal model according to FIG. 4A (without mandible).

FIG. 6b is a representation of the target structure and of the normal model according to FIG. 6a, but with a normal model partially adapted to the target structure.

FIG. 6c is a representation of the target structure and of the normal model according to FIG. 6b, but with a normal model further adapted to the target structure.

FIG. 7 is a representation of anatomical markers on a skull normal model according to FIG. 4A.

FIG. 8 is a representation of a surface model of a human pelvis formed on a triangle basis as used in accordance with the invention.

### DESCRIPTION OF THE PREFERRED EMBODIMENTS

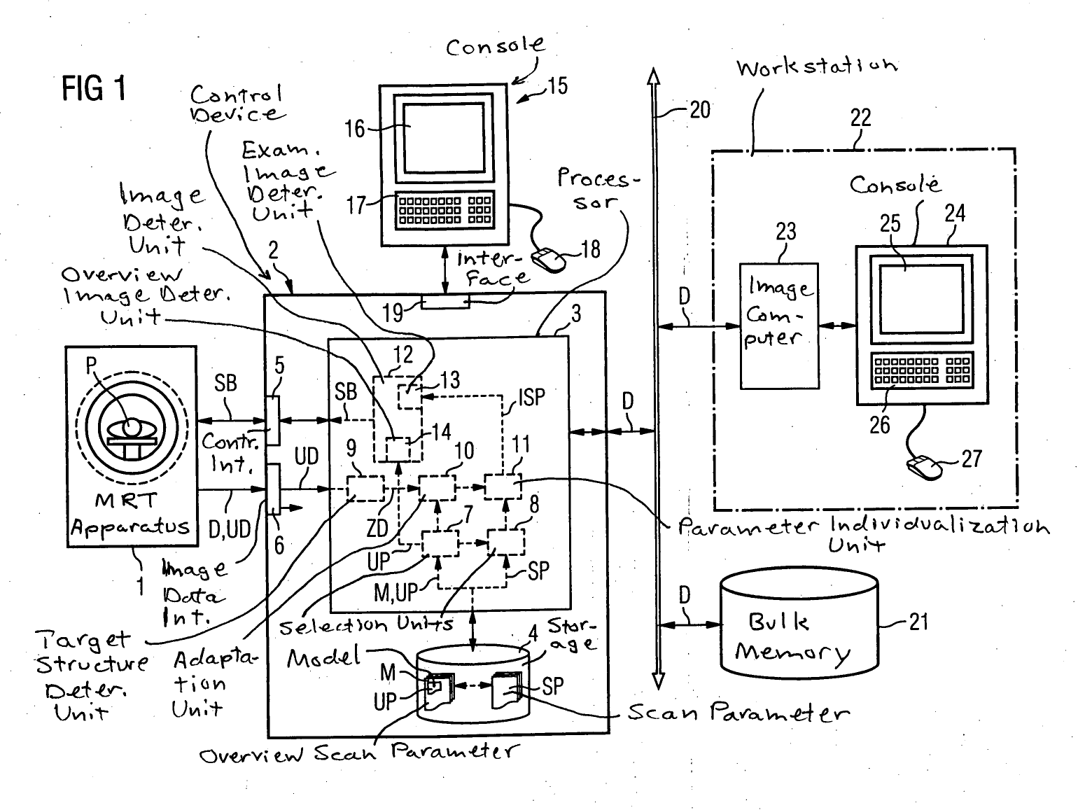

In the exemplary embodiment shown in FIG. 1, the inventive magnetic resonance tomography apparatus 1 is connected with an associated inventive control device 2 via a bus 20. Further components such as, for example, a bulk memory 21 for storage of image data D and a workstation 22 are connected to the bus 20. This workstation 22 is formed of an image computer 23 and a console 24 which, as is typical, has a screen 25, a keyboard 26 and a pointer device, for example a mouse 27. The workstation 22 serves, for example, for observation and processing of the images generated by the MRT apparatus 1.

Naturally, other components that are present in a typical radiological information system (RIS), for example further modalities, bulk memories, workstations, output devices (such as printers, filming stations or similar) can also be connected to the bus 20 to form a larger network. A connection with an external network or with further RIS's is likewise possible. All data are preferably formatted according to the known DICOM standard (Digital Imaging and Communication in Medicine) for communication among the individual components.

In the shown exemplary embodiment, the control device 2 is accommodated in a separate device, i.e., a computer with a programmable processor on which the control software for activation of the MRT apparatus 1 runs. Via a control interface 5, the control device 2 transmits control commands SB to the MRT apparatus 1 so that the desired measurement is implemented.

Various image data D, UD are accepted from the MRT apparatus 1 via an image data interface 6 and then further processed within the control device 2. In order to be able to operate the control device 2 directly on site, via an interface 19 a console 15 is connected which has as a user interface, a screen 16, a keyboard 17 and a pointing device, for example a mouse 18. Alternatively, it is possible for the operation to ensue, for example, via the workstation 22 likewise connected to the network 20 instead of via the console 15 directly connected to the control device 2. For this, the workstation 22 can also be located in the immediate spatial proximity of the apparatus 1.

The control device 2 alternatively can be an integrated component of the MRT apparatus 1. The console 15 also can be an integrated component of the control device 2 or of the MRT apparatus 1, such that all components are combined into one apparatus.

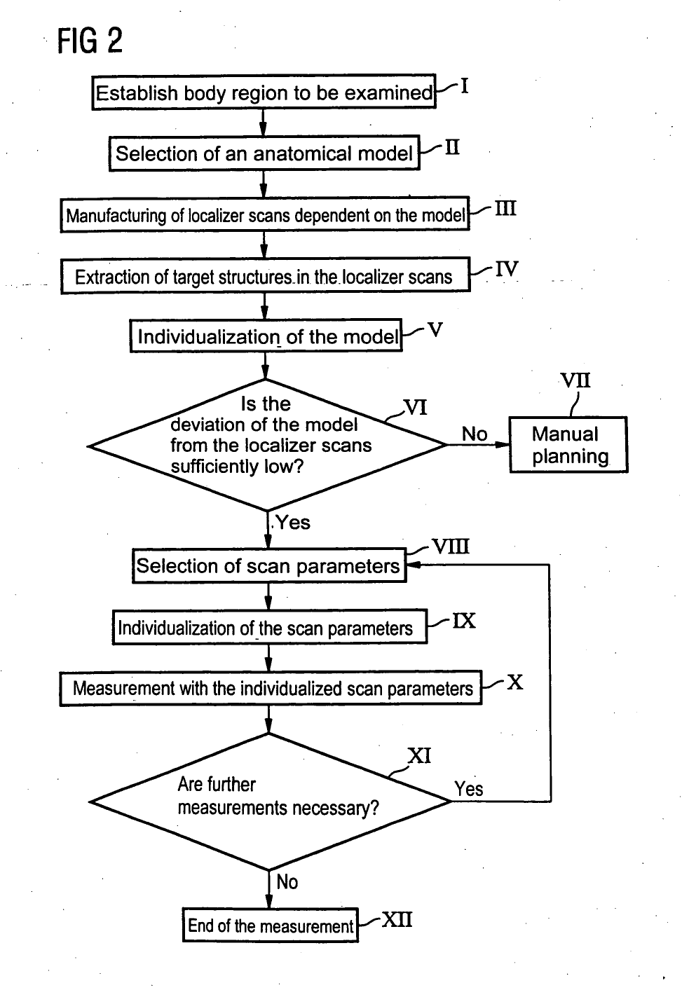

A possible procedure executed by the inventive method for automatic control of the MRT apparatus 1 during measurements is shown in FIG. 2.

Initially, in a first method step I the body region to be examined is established and the patient P is correspondingly positioned in the magnetic resonance tomography apparatus 1 or a suitable local coil is positioned on the patient P. Thus, for example, the head of the patient P is brought into a head coil or the like in an examination of the base of the skull.

As a second method step II, initially an appropriate anatomical model M (in the cited example of the head examination a skull model) is selected from a databank. A storage device 4, in which is stored a databank with a wide variety of models M, is shown in FIG. 1 as an integrated component 1 of the control device 2.

The selection of the model M ensues with a first selection unit 7 which is realized here in the form of a software module on the processor 3 of the control device 2. The input of the diagnostic question by the operator ensues, for example, via the console 15.

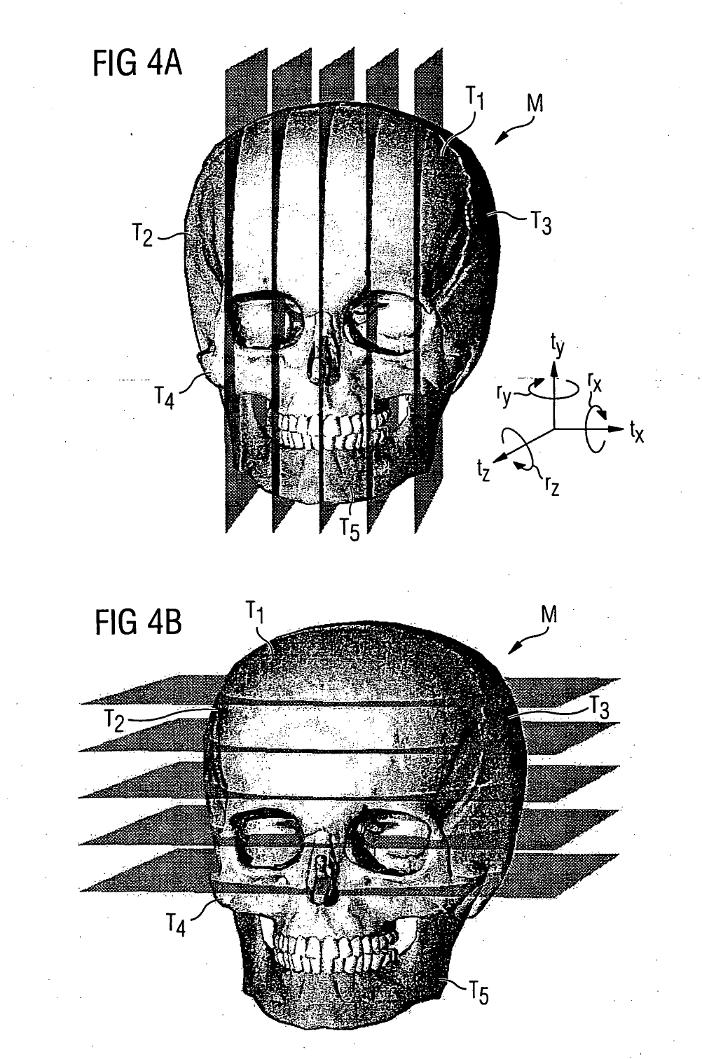

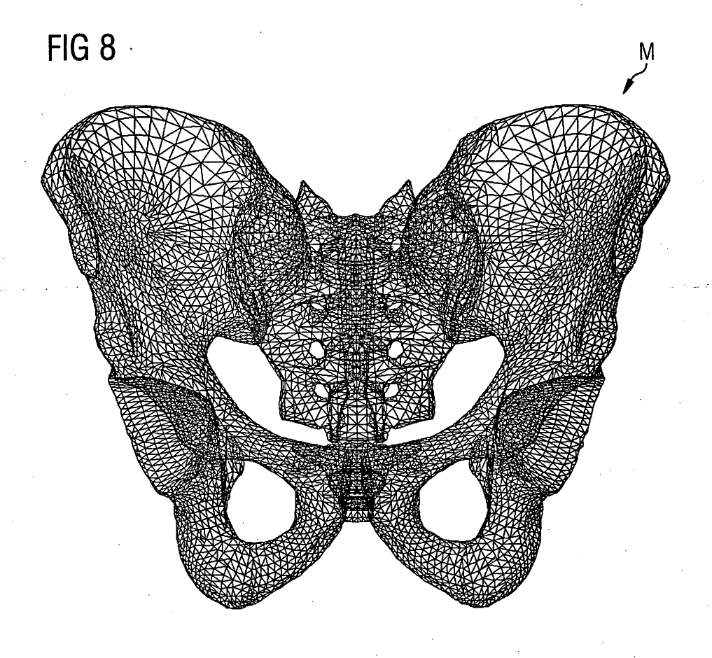

The normal models M can also be models that are composed of a number of partial subjects. Thus, for example, a knee model is comprised of the model parts “femur”, “tibia”, “patella” (kneecap and the individual menisci). In contrast, given a diagnostic inquiry with respect to the head of the patient in order, for example, to verify a suspicion of a skull fracture, a cranial bone normal model is necessary. FIGS. 4A and 4B show a possible skull normal model M which, among other things, is composed of model parts (recognizable in FIGS. 4A and 4B) frontal bone T₁, right parietal bone T₂, left parietal bone T₃, visceral cranium T₄ and mandible T₅. Further model parts that are not shown in FIGS. 4A and 4B are the occipital bone and the base of the skull. For better recognizability, the model is shown with a continuous surface in FIGS. 4A and 4B. The models are additionally, preferably assembled based on triangles. A surface model of a pelvis based on triangles is shown in FIG. 8.

In step III, acquisition of overview images (localizer scans) ensues dependent on the selected model. The overview scan parameters UP on the basis of which the overview images are obtained are stored together with the model M. This means that, given selection of the model M, it is simultaneously established which and how many overview images are generated. Candidate slice image planes for overview images are indicated in FIGS. 4A and 4B, whereby FIG. 4A contains sagittal slice planes and FIG. 4B contains transversal slice planes. For better clarity, only five slice planes are indicated with a very large separation from one another. In reality, the slice planes are significantly denser.

Since the overview images here are used not only for conventional manual graphical planning of the MR examination, but also for individualization of anatomical models, higher quality demands are placed on the images. In addition to the image quality, the slice count, the slice separation and the image field are sometimes also relevant. In contrast to this, for the most part it is not necessary that the overview slice images have a precise defined position with regard to the examination subject. It is adequate that, with the overview images, sufficient data are obtained to determine the target structure, such that subsequently a precise adaptation of the normal model can ensue. This means that it is often largely insignificant whether—as shown in FIGS. 4A and 4B using the skull model—the slice image data are acquired transversally, sagittally or diagonally, as long as sufficient sampling points are later available for the individualization of the model in the target structure. If applicable, the acquisition of images under different directions also can be done.

The various overview scan parameters UP determine to a high degree the database for the later individualization algorithm. In order to ensure a stable method execution in the individualization, these overview scan parameters UP are experimentally determined for each model M (preferably in the foreground via examinations of a larger region) and then linked with the appertaining model M, preferably in the form of a complete localizer protocol. Given selection of a model, the overview scan parameters UP are transferred to an image determination unit 12 likewise realized in the form of software in the processor 3. This image determination unit 12 converts the measurement protocols or the various scan parameters—and thus also the overview scan parameters—into control commands SB that are then transferred via the control interface 5 to the MRT apparatus 1, so that there the appropriate measurement sequences are directed in the correct series. In the present example, the image determination unit has, as a subroutine, a separate overview image determination unit 14 which serves to generate the control commands SB for measurement of the overview images on the basis of the overview scan parameters UP. Another routine is the examination image determination unit 13 which serves to generate, using further scan parameters, the control commands SB for implementation of the actual measurement for examination of the patient P.

The overview image data UD generated in the overview scans are then (like all remaining image data D) transferred via the image data interface 6 by the control unit 2 and further processed there.

A target structure Z is thereby determined within the overview slice image data UD in a method step IV, dependent on the predetermined diagnostic question. This preferably ensues completely automatically with the aid of the aforementioned contour analysis. Given specific structures and specific acquisition methods, a threshold method can also be used, as has been described above. In the exemplary embodiment shown in FIG. 1, this determination of the target structures Z ensues within a target structure determination unit 9 likewise realized in the form of software on the processor 3. This relays the target structure ZD to an adaptation unit 10 likewise realized in the form of software, which moreover contains the data about the model M from the selection unit 7.

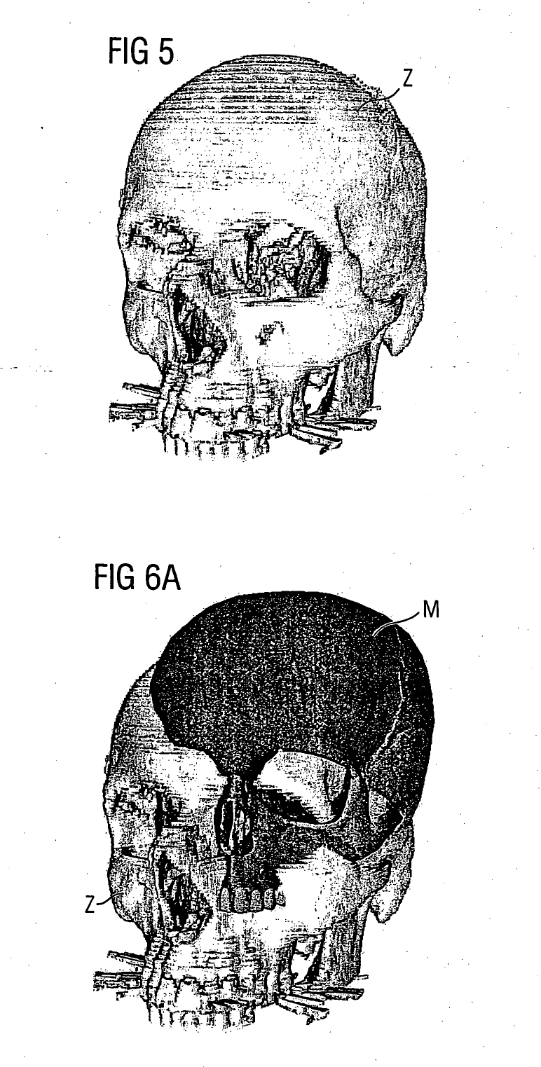

An individualization of the model M then ensues in the adaptation unit 10 in the method step V, i.e. the normal model M is adapted to the determined target structure Z. A target structure Z for a skull examination, which could have been acquired from overview image data of a patient, is shown in FIG. 5. This target structure can serve, for example, for adaptation of the normal model according to FIGS. 4A and 4B.

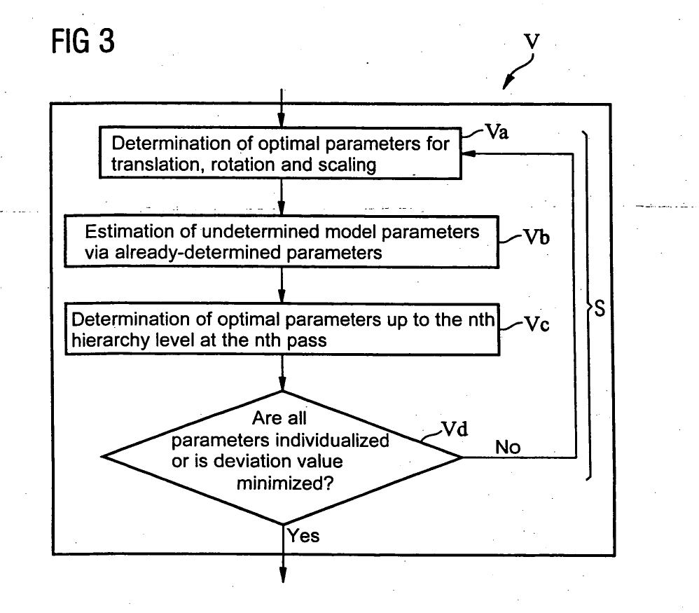

A preferred embodiment of the individualization process is schematically shown more defined in FIG. 3 in the form of a flow chart.

In this adaptation process, the individual model parameters are varied in a series of iteration steps S until ultimately all parameters are individualized or the individualization is sufficient, meaning that the deviation between the normal model M and the target structure Z is minimal or lies below a predetermined threshold. Each iteration step S includes a number of process steps Va, Vb, Vc, Vd that are traversed in a loop.

The first iteration step S begins with the method step Va, in which initially the optimal parameters are determined for the translation, rotation and scaling. These are the parameters of the uppermost (in the following “0th”) hierarchy class, since these parameters affect the overall geometry. The three parameters of the translation tₓ, t_{y} t_{z} and the three parameters of the rotation rₓ, r_{y}, r_{z} around the three model axes are schematically indicated in FIG. 4A.

If this adaptation ensues as far as possible, in a further step Vb any still unadjusted model parameters are estimated by already-determined parameters. This means that starting values for subordinate parameters are estimated from the settings of superordinate parameters. An example of this is the estimation of the knee width from the settings of a scaling parameter for the body size. This value is predetermined as an initial value for the subsequent adjustment of the appertaining parameter. In this manner, the method can be significantly accelerated. In the method step Vc the appertaining parameters are then optimally adjusted.

In the exemplary embodiment, the parameters are hierarchically arranged with regard to their influence on the anatomical overall geometry of the model. The greater the geometric effect of a parameter, the higher it stands in the hierarchy. With an increasing number of the iteration steps S, the number of the adjustable model parameters increases corresponding with the hierarchical order.

This means that, in the first pass of the loop, in the step Vc only the parameters of the 1st hierarchy level below the 0th hierarchy level are used for adjustment of the model. In the second pass, it is then possible to first re-subject the model to a translation, rotation and scaling again in the method step Va. Subsequently, in the method step Vb the still-undetermined model parameters of the 2nd hierarchy class are estimated using already-determined parameters, that are then added for adjustment in step Vc. This method is then repeated n times. In the nth iteration step all parameters of the nth level are optimized, and in the last step Vd of the iteration step S it is in turn checked whether still further parameters are available that have not been previously optimized. A new, (n+1)-th iteration step subsequently begins, and the model M is correspondingly newly shifted, rotated or scaled, and finally the series can be adjusted again according to all parameters, and now the parameters of the (n+1)-th class are also available. In the method step Vd, it is subsequently re-checked whether all parameters are individualized, i.e. whether parameters still exist that have not yet been optimized, or whether the desired adaptation has been achieved.

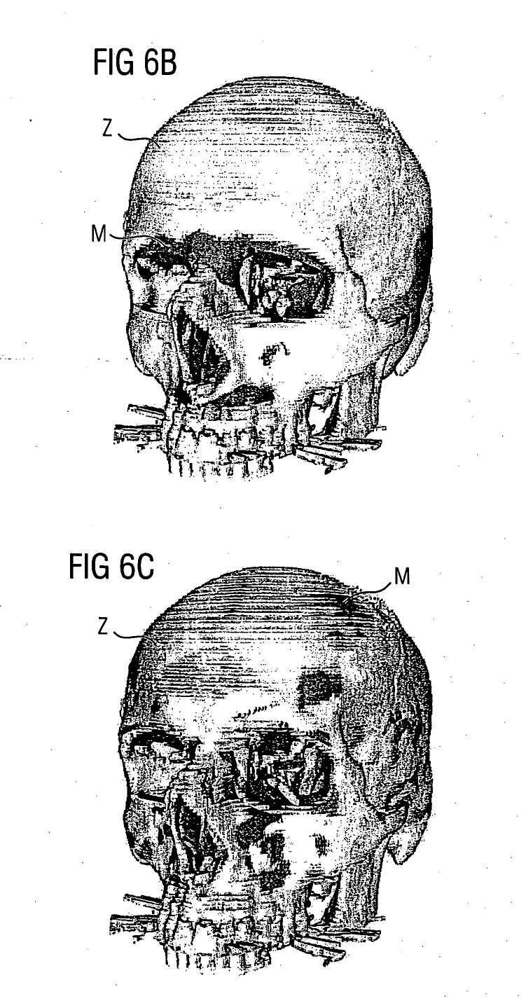

FIGS. 6A through 6C show a very simple case for such an adaptation process. In FIGS. 6A-6C, the model M is again shown as a continuous surface for better clarity. FIG. 6A shows the target structure Z with the shifted model M. Via a simple translation, rotation and scaling, the image shown in FIG. 6B, in which the model M is already relatively well adapted to the target structure Z, is then achieved. Ultimately, the adaptation achieved in FIG. 6C is obtained by an adjustment of further subordinate parameters.

Using the iteration method described above, an optimally timesaving and effective adaptation ensues. The target structure Z and the associated model M as well as current calculated deviation values, or the current calculated value of a deviation function, can be shown on the screen 6 of the console 5 at any time during the adaptation. Moreover, the deviations can also be visualized as shown in FIGS. 6A through 6C. The visualization of the deviation can additionally ensue with suitable coloration.

The subordinate hierarchy classes result from the quantitative analysis of the geometric influence. For this purpose, each parameter is modified and the resulting deviation of the geometrically modified model from the initial state is calculated. This deviation can be quantified, for example, by the sum of the geometric separations of corresponding model triangles when surface models based on triangles (as shown in FIG. 8) are used. By specification of numerical intervals for the deviation, the parameters then can be classified into the hierarchy classes. This is dependent on, among other things, the width of the numerical intervals for the deviations. As explained above, these parameters in the same hierarchy class are simultaneously offered for modification for the first time within a determined iteration step S, or are automatically modified in an automatic adaptation step.

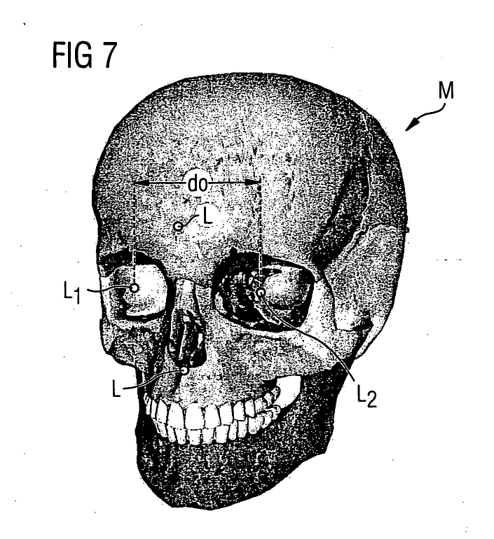

As already mentioned, in this method model parameters preferably are used that are directly connected with one or more positions of specific anatomical landmarks of the model. Examples of such parameters are the positions of the anatomical landmarks L, L1, L2 indicated on a skull model in FIG. 7 or the distances between the individual landmarks, like the distance d0 between the anatomical landmarks L1, L2 in the center of the orbital sockets (eye sockets). In order to adjust this separation d0 of the orbital sockets given a manual intervention of an operator in the automatic adaptation process, the user can select one of the anatomical landmarks (for example by means of a mouse pointer) and interactively modify its position. The geometry of the model M is then automatically appropriately deformed as well.

In a variation of a model parameter exhibiting a separation between two anatomical landmarks of the normal model M, the geometry of the normal model is preferably deformed proportional to the separation change in a region along a straight line between the anatomical landmarks. Given a variation of a model parameter exhibiting a modification of the position of a first anatomical landmark relative to an adjacent landmark, the geometry of the normal model M preferably is appropriately deformed as well in the direction of the appertaining, adjacent landmarks in a surrounding area around the appertaining first landmark. The deformation decreases with increasing separation from the appertaining first anatomical landmarks. This means that the deformation is more significant in a narrower region around the landmark than in the regions spaced further from it in order to achieve the effect shown in the figures. Other transformation rules are also possible, insofar as they lead to anatomically reasonable transformations. This is, if applicable, dependent on the respectively selected model.

Using the anatomical markers L, L1, L2 on the skull model in FIG. 8, a typical example can be explained in which the separations between two landmarks are classified in different hierarchy classes. Thus the skull model shown in FIG. 8 is determined not only by the separation d0 of both orbital sockets but also is parameterized by the separation of both Processi styloidei, which are small boney appendages on the skull base (not recognizable in the perspective in FIG. 8). Here the geometric effect of the first parameter, which specifies the orbital separation, is greater than the geometric effect of the second parameter, which specifies the separation between the Processi styloidei. This can be examined, for example, by means of a geometry modification of the model given a parameter modification by one millimeter. Since the Processi styloidei are relatively small structures, the geometric model modification is limited to a small region around these bone appendages. In contrast to these are the relatively very large orbital sockets. Given a modification of the orbital separation, a multiple portion of the model's geometry will be modified and this will lead to an increased deviation. The parameter of the orbital separation is therefore in a significantly higher hierarchy class than the modification of the separation of the Processi styloidei, since in principle parameters with a greater geometric scope of the parameter hierarchy and higher than parameters with a more local effect.

Finally, if all adjustable parameters have been individualized or if the deviation function has achieved its minimal value, in method step VI it is checked whether the deviation of the individualized normal model from the data set (i.e. the target structure) is sufficiently small. It can be checked, for example, whether the currently achieved deviation value is below a limit value. If this is not the case, the automatic process is terminated and the further processing ensues—as schematically shown as a method step VII—in a conventional manner. This means that the overview image data are then used by the operator for manual adjustment of the further scan parameters. In the case of such a termination a signal is output to the operator such that the operator immediately recognizes that the operator must manually conduct the process further.

If, in contrast, the adaptation of the normal model M to the target structure Z is sufficient, for the further examination a selection of scan parameters SP corresponding to the anatomical normal model M and corresponding to the diagnostic question can then ensue in the method step VIII. The selection of the various scan parameters SP ensues via a second selection unit 8 which—as schematically shown in FIG. 1—is preferably likewise realized in the form of software on the processor 3 of the control device 2. This second selection unit 8 contains, for example, the model information from the first selection unit 7. The information about the diagnostic inquiry has previously been entered by the operator at the console 15, or the operator has already selected a diagnostic inquiry from various predetermined diagnostic questions.

The selection of scan parameters SP dependent on the diagnostic inquiry can return to the selection of a suitable examination protocol, by the scan parameters being combined for a specific MR examination. Certain protocols depict the general morphology. This concerns, for example, the T1, T2 as well as PD protocols. In contrast, other protocols depict specific morphologies. Thus, for example, blood vessels are shown by 3D gradient echo protocols using MR contrast agents. The diffusion and perfusion imaging on the basis of EPI protocols enables the targeted examination of encephalopathies (brain diseases). In general, there is a range of examination protocols for most different diagnostic inquiries. The protocol parameters separate into specific scan parameters only for the corresponding protocol and general scan parameters. Of particular importance are the always-necessary geometric scan parameters which must be individually adjusted for the respective concrete examination case. Thus in the MR examinations it is necessary that the corresponding slice packets be positioned and aligned. In addition, in most cases the slice separation and the slice thickness also must be individually selected, in the context of a rectangular image field. The goal of this individual scan parameter adjustment is the standardized reproduction of the clinically relevant anatomical structures. The slice packets are pre-aligned to anatomical landmarks. An example of this is a knee examination in which the easily recognizable joint cavity is used, or in brain examinations using the front and rear commissures. For example, the position and orientation of a scan plane are normally defined by the specification of at least three support points. The delimitation of the scan volume also can be associated with the anatomical model by suitable support points, whereby among other things the image field is established. According to the invention, this alignment and adjustment of the individual scan parameters no longer ensues during the measurement, but instead ensues once on the normal model suitable for the diagnostic inquiry. For this purpose, finished protocols that also include the geometric scan parameters for the appertaining normal model are associated with each model for each of the possible questions.

The scan parameters are stored in connection with the respective model, for example in a databank. In FIG. 1, this is schematically shown as the storage unit 4 of the control device 2. The storage structure can be designed, for example, as a type of tree structure, such that various diagnostic inquiries are associated with each model and in turn the associated scan parameters are associated with these diagnostic inquiries.

The geometric scan parameters SP selected by the second selection unit 8 in the method step VIII consequently correspond initially to the selected normal models, i.e. they are “normal scan parameters”. Consequently, an individualization of the normal scan parameters SP must ensue corresponding to the individualized normal model which has been adapted by the adaptation unit 10 to the target structure in the overview image data, which individualization occurs in method step IX by means of a parameter individualization unit 11 which is preferably realized in the form of software on the processor 3. The information about the 3D transformation implemented for adaptation of the normal model to the target structure Z or about the individualization algorithm used receives the parameter individualization unit 11 from the adaptation unit 10 and can thus implement the corresponding individualization of the scan parameters SP. For example, in the parameter individualization unit 11, for adaptation of a scan plane the support points which map out the scan plane with regard to the anatomical normal model M are transformed and thus individualized corresponding to the three-dimensional transformation of the normal model M.

The individualized scan parameters ISP are then forwarded to the examination image determination unit 13. This then converts the individualized scan parameters ISP into corresponding control commands SB for the MRT apparatus 1, such that the desired measurement is implemented in the method step X.

Optionally, in the method step XI it can then be established whether further measurements are necessary. This can ensue manually, i.e. according to a corresponding pre-diagnosis by a trained operator of the MRT apparatus 1, or can ensue (if applicable) completely automatically via an automatic image evaluation. A jump back to the method step VIII then ensues in the method procedure corresponding to the determination of whether and which further measurements are necessary, and scan parameters are again selected for the respective model dependent on the further diagnostic question, and the method steps IX, X, and XI are executed again.

If it is established that no further measurements are necessary, in method step XII the measurement is finally ended and the acquired image data D can be sent, for example, be sent over the bus 20 and be stored in the bulk memory 21, or can be transferred to other workstations for further processing or viewing, or can be transferred to other image observation units for further diagnosis by a radiologist. Likewise, it is possible to send the image data to filming stations or similar in order to generate films or other printouts.

It should again be noted that the system architectures and processes shown in the figures are only exemplary embodiments that can be modified in terms of detail by those skilled in the art. In particular, it is possible for the components of the control device 2 to be realized not on a processor but rather on various processors networked among one another. Likewise it is naturally possible for the components to be realized on different computers networked with one another. Thus particularly computationally intensive processes such as the individualization of the model can be sourced out to suitable computers which then deliver back only the end result.

The inventive method and apparatus can be used to upgrade or retrofit existing control devices or magnetic resonance tomography apparatuses in which known post-processing processes are completely implemented. In many cases, if applicable an update of the control software with suitable control software modules is also sufficient.

Although modifications and changes may be suggested by those skilled in the art, it is the intention of the inventor to embody within the patent warranted hereon all changes and modifications as reasonably and properly come within the scope of his contribution to the art.

1. A method for operating a magnetic resonance tomography apparatus, comprising the steps of:
for an examination subject to be examined dependent on a diagnostic inquiry, selecting an anatomical model having a variable geometry and varying the geometry dependent on the diagnostic inquiry;
acquiring a plurality of magnetic resonance overview images of a region of the examination subject, and setting scan parameters for said overview images dependent on the selected anatomical normal model, said overview images each being composed of slice image data;
determining a target structure in the slice image data of the respective overview images;
individualizing said anatomical normal model dependent on said target structure, thereby obtaining an individualized anatomical normal model;
selecting scan parameters for controlling said magnetic resonance scanner for obtaining diagnostic magnetic resonance images of the region of the examination subject dependent on said selected anatomical normal model and said diagnostic inquiry;
individualizing the selected scan parameters dependent on said individualized normal anatomical model, thereby obtaining individualized scan parameters; and
controlling said magnetic resonance scanner to obtain said diagnostic magnetic resonance images of the region of the examination subject, using said individualized scan parameters.

2. A method as claimed in claim 1 comprising, after individualizing said anatomical normal model, checking whether a deviation, if present, of said individualized normal anatomical model from said target structure is below a predetermined limit value, and otherwise terminating individualization of said anatomical normal model.

3. A method as claimed in claim 1 comprising storing a plurality of different anatomical normal models together with overview scan parameters respectively associated therewith, selecting said selected anatomical normal model from among the plurality of stored anatomical normal models, and using the scan parameters associated with the selected normal anatomical model for acquiring said overview images.

4. A method as claimed in claim 1 comprising employing parameters, as said scan parameters, setting a position, number and type of said overview images.

5. A method as claimed in claim 1 comprising individualizing said normal anatomical model by employing model parameters to generate successive modified normal anatomical models and, for each modified normal anatomical model, representing a deviation thereof from said target structure as a deviation function, and automatically modifying said model parameters to minimize said deviation function.

6. A method as claimed in claim 1 comprising individualizing said normal anatomical model in a plurality of iteration steps using model parameters, and ordering said slice image data of said overview images hierarchically with regard to an influence of the slice image data on said geometry of said anatomical normal model, and increasing a number of adjusted model parameters dependent on the hierarchical ordering with an increasing number of said iteration steps.

7. A method as claimed in claim 6 comprising respectively associating said model parameters in different hierarchy classes.

8. A method as claimed in claim 7 comprising associating the respective model parameters with said hierarchy classes dependent on a deviation of said geometry of said normal anatomical model that occurs when the model parameter is varied by a predetermined value.

9. A method as claimed in claim 8 comprising associating specific value ranges of said deviation with the perspective hierarchy classes.

10. A method as claimed in claim 1 comprising employing a surface model generated on a triangle basis as said normal anatomical model.

11. A method as claimed in claim 1 comprising individualizing said normal anatomical model by modifying model parameters of the normal anatomical model, and respectively linking the model parameters with a position of at least one anatomical landmark of said examination subject, to produce a modified normal anatomical model with a set of said model parameters exhibiting an anatomically sensible geometry.

12. A method as claimed in claim 1 comprising determining said target structure from said slice image data of said overview images at least partially automatically using a contour analysis technique.

13. A method as claimed in claim 1 comprising automatically classifying the examination subject dependent on further slice images acquired of the examination subject.

14. A computer program product loadable into a control unit of a magnetic resonance tomography apparatus having a magnetic resonance scanner, for controlling said magnetic resonance tomography apparatus to:
select, for an examination subject to be examined dependent on a diagnostic inquiry, an anatomical model having a variable geometry and varying the geometry dependent on the diagnostic inquiry;
acquire a plurality of magnetic resonance overview images of a region of the examination subject, and setting scan parameters for said overview images dependent on the selected anatomical normal model, said overview images each being composed of slice image data;
determine a target structure in the slice image data of the respective overview images;
individualize said anatomical normal model dependent on said target structure, thereby obtaining an individualized anatomical normal model;
select scan parameters for controlling said magnetic resonance scanner for obtaining diagnostic magnetic resonance images of the region of the examination subject dependent on said selected anatomical normal model and said diagnostic inquiry;
individualize the selected scan parameters dependent on said individualized normal anatomical model, thereby obtaining individualized scan parameters; and
control said magnetic resonance scanner to obtain said diagnostic magnetic resonance images of the region of the examination subject, using said individualized scan parameters.

15. A control device for operating a magnetic resonance tomography apparatus having a scanner, said control device, for an examination subject to be examined dependent on a diagnostic inquiry, selecting an anatomical model having a variable geometry and varying the geometry dependent on the diagnostic inquiry, operating said magnetic resonance scanner to acquire a plurality of magnetic resonance overview images of a region of the examination subject, with said control device setting scan parameters for said overview images dependent on the selected anatomical normal model, said overview images each being composed of slice image data, determining a target structure in the slice image data of the respective overview images, said control device individualizing said anatomical normal model dependent on said tar get structure, thereby obtaining an individualized anatomical normal model, selecting scan parameters for controlling said magnetic resonance scanner for obtaining diagnostic magnetic resonance images of the region of the examination subject dependent on said selected anatomical normal model and said diagnostic inquiry, individualizing the selected scan parameters dependent on said individualized normal anatomical model, thereby obtaining individualized scan parameters, and controlling said magnetic resonance scanner to obtain said diagnostic magnetic resonance images of the region of the examination subject, using said individualized scan parameters.

16. A magnetic resonance tomography apparatus comprising:
a magnetic resonance scanner adapted to receive an examination subject therein; and
a control device for operating said magnetic resonance scanner, said control device, for an examination subject to be examined dependent on a diagnostic inquiry, selecting an anatomical model having a variable geometry and varying the geometry dependent on the diagnostic inquiry, operating said magnetic resonance scanner to acquire a plurality of magnetic resonance overview images of a region of the examination subject, with said control device setting scan parameters for said overview images dependent on the selected anatomical normal model, said overview images each being composed of slice image data, said control device determining a target structure in the slice image data of the respective overview images, individualizing said anatomical normal model dependent on said target structure, thereby obtaining an individualized anatomical normal model, selecting scan parameters for controlling said magnetic resonance scanner for obtaining diagnostic magnetic resonance images of the region of the examination subject dependent on said selected anatomical normal model and said diagnostic inquiry, individualizing the selected scan parameters dependent on said individualized normal anatomical model, thereby obtaining individualized scan parameters, and controlling said magnetic resonance scanner to obtain said diagnostic magnetic resonance images of the region of the examination subject, using said individualized scan parameters.
